(19) United States
(12) Patent Application Publication
(10) Pub. No.: US 2026/0311887 A1
(43) Pub. Date: Oct. 8, 2026

(54) Cognitive decline identification system and method

(71) Applicant: Don Roosan, Lawrence, MA (US)

(72) Inventor: Don Roosan, Lawrence, MA (US)

(21) Appl. No.: 19/635,088

(22) Filed: Mar. 31, 2026

Related U.S. Application Data
(60) Provisional application No. 63/784,205, filed on Apr. 6, 2025.

(51) Int. Cl. G16H 50/30; G16H 15/00; G16H 50/20

(52) U.S. Cl. CPC G16H 50/30; G16H 15/00; G16H 50/20

(57) ABSTRACT

Cognitive decline identification system and method. The method includes presenting, via a user interface executing on a digital device, a prompt for a user to provide a first drawing input of an object; receiving, via the user interface, a first drawing input from the user in response to the prompt; analyzing the first drawing input by implementing a machine learning procedure to determine whether the first drawing input satisfies an objective criterion; and calculating a cognitive assessment score based on the analysis of the first drawing input, wherein the cognitive assessment score indicates a likelihood of cognitive decline of the user.

### CROSS REFERENCE TO RELATED APPLICATIONS

The present application claims the benefit of and priority to co-pending U.S. provisional Application No. 63/784,205, filed on Apr. 6, 2025, the content of which is hereby incorporated by reference as if set forth in its entirety herein.

### TECHNICAL FIELD

Embodiments described herein generally relate to systems and methods for diagnosing medical conditions and, more particularly but not exclusively, to systems and methods for identifying cognitive decline.

### BACKGROUND

Early detection of cognitive impairment is crucial for intervening, managing, and improving the quality of life for patients and their families. Existing cognitive assessment tools fail to adequately detect early-stage cognitive decline. This may be due to language barriers or dependencies, cultural bias, reliance on manual scoring, or some combination thereof.

Failure to detect cognitive decline at an early stage may lead to complications as the condition worsens. For example, late detection inherently results in patients being diagnosed at advanced stages. Treatment options at late stages are less effective, and the opportunity to slow disease progression is diminished.

A need exists, therefore, for systems and methods that overcome the disadvantages associated with existing techniques.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a method for identifying cognitive decline. The method includes presenting, via a user interface executing on a digital device, a prompt for a user to provide a first drawing input of an object; receiving, via the user interface, a first drawing input from the user in response to the prompt; analyzing the first drawing input by implementing a machine learning procedure to determine whether the first drawing input satisfies an objective criterion; and calculating a cognitive assessment score based on the analysis of the first drawing input, wherein the cognitive assessment score indicates a likelihood of cognitive decline of the user.

In some embodiments, analyzing the first drawing input comprises evaluating at least one of visuospatial ability, semantic memory, or executive function based on characteristics of the first drawing input.

In some embodiments, the method further includes generating a report providing an explanation of the calculated cognitive assessment score. In some embodiments, the generated report includes a highlighted portion of the first drawing input and an annotation accompanying the highlighted portion.

In some embodiments, the method further includes generating a treatment plan for the user based on the calculated cognitive assessment score.

In some embodiments, analyzing the first drawing input includes detecting an error in the first drawing input and categorizing the detected error, wherein the calculated cognitive assessment score is based on the category of the detected error. In some embodiments, the method further includes generating a treatment plan based on the category of the detected error.

In some embodiments, analyzing the first drawing input includes presenting a model of the object to the user via the user interface, and detecting conformance of the first drawing input to the model, wherein the generated cognitive assessment score is based at least in part on the conformance of the first drawing input to the model.

In some embodiments, the method further includes presenting at least a second prompt for the user to provide a second drawing input of a second object; receiving a second drawing input from the user; analyzing the second drawing input, wherein each of the first drawing input and the second drawing input are designed to test one or more of visuospatial ability, semantic memory, or executive function based on characteristics of each drawing input.

According to another aspect, embodiments relate to a system for identifying cognitive decline. The system includes a user interface executing on a digital device and configured to present a prompt for a user to provide a first drawing input of an object, and receive a first drawing input from the user in response to the prompt; and one or more processors executing instructions stored on memory to analyze the first drawing input by implementing a machine learning procedure to determine whether the first drawing input satisfies an objective criterion, and calculate a cognitive assessment score based on the analysis of the first drawing input, wherein the cognitive assessment score indicates a likelihood of cognitive decline of the user.

In some embodiments, wherein the one or more processors analyze the first drawing input by evaluating at least one of visuospatial ability, semantic memory, or executive function based on characteristics of the first drawing input.

In some embodiments, the one or more processors are further configured to generate a report providing an explanation of the calculated cognitive assessment score. In some embodiments, the generated report includes a highlighted portion of the first drawing input and an annotation accompanying the highlighted portion.

In some embodiments, the one or more processors are further configured to generate a treatment plan for the user based on the calculated cognitive assessment score.

In some embodiments, the one or more processors analyze the drawing input by detecting an error in the drawing input, and categorizing the detected error, wherein the calculated cognitive assessment score is based on the category of the detected error. In some embodiments, the one or more processors are further configured to generate a treatment plan for the user based on the category of the detected error.

In some embodiments, the one or more processors analyze the drawing input by presenting a model of the object to the user via the user interface, and detecting conformance of the first drawing input to the model, wherein the generated cognitive assessment score is based at least in part on the conformance of the first drawing input to the model.

In some embodiments, the user interface is further configured to present at least a second prompt for the user to provide a second drawing input of a second object, and receive a second drawing input from the user, and the one or more processors are further configured to analyze the second drawing input, wherein each of the first drawing input and the second drawing input are designed to test one or more of visuospatial ability, semantic memory, or executive function based on characteristics of each drawing input.

According to yet another aspect, embodiments relate to a computer program product for identifying cognitive decline. The computer program product comprising computer executable code embodied in one or more non-transitory computer readable media that, when executing on one or more processors, performs the steps of: presenting, via a user interface executing on a digital device, a prompt for a user to provide a first drawing input of an object; receiving, via the user interface, a first drawing input from the user in response to the prompt; analyzing the first drawing input by implementing a machine learning procedure to determine whether the first drawing input satisfies an objective criterion; and calculating a cognitive assessment score based on the analysis of the first drawing input, wherein the cognitive assessment score indicates a likelihood of cognitive decline of the user.

In some embodiments, analyzing the first drawing input comprises evaluating at least one of visuospatial ability, semantic memory, or executive function based on characteristics of the drawing input.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a system for identifying cognitive decline in accordance with one embodiment;

FIG. 2 illustrates an imagery analysis module in accordance with one embodiment;

FIG. 3 illustrates an imagery analysis module in accordance with another embodiment;

FIG. 4 illustrates an imagery analysis module in accordance with another embodiment;

FIG. 5 illustrates a user interface presenting a summary of a patient's drawing input in accordance with one embodiment; and

FIG. 6 depicts a flowchart of a method for identifying cognitive decline in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Cognitive decline is a pressing global issue. Over 55 million people are estimated to be currently living with dementia worldwide, and this number is projected to increase to 139 million by 2050. Detecting cognitive impairment in its early stages is critical for effectively intervening and managing a patient's cognitive decline. Early detection can improve the quality of life for patients and their families. However, existing detection tools are inadequate to detect cognitive decline due to, for example, language dependence, cultural bias, reliance on manual scoring techniques, or some combination thereof.

The present application provides novel techniques for identifying cognitive decline. The disclosed embodiments administer one or more drawing assessment tests to a patient to identify whether, and to what extent, the patient is suffering from cognitive decline. Depending on the type of error(s) the patient makes in creating the drawing(s), the embodiments can discern the severity of and type of decline.

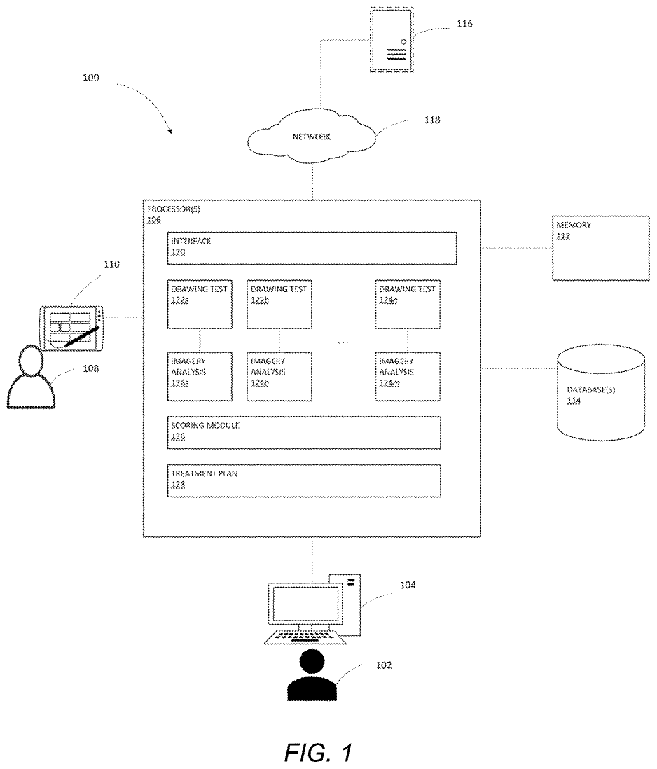

FIG. 1 illustrates a system 100 for identifying cognitive decline in accordance with one embodiment. In operation, a user 102 may access a user device 104 in operable communication with one or more processors 106. The user 102 may include medical personnel tasked with administering one or more tests to a patient 108 to identify any cognitive decline of the patient 108. The patient 108 may access a patient device 110 to perform the administered test(s).

The processor(s) 106 may be in further communication with or otherwise in operable connectivity with memory 112, one or more database(s) 114, and one or more servers 116 over a network 118.

The processor(s) 106 may include an interface 120 configured to receive data regarding tests from the database 114 and servers 116. The interface 120 may also communicate instructions regarding tests to be administered to the patient 108 via the patient device 110. The interface 120 may be configured as a wired connection such as through Ethernet, Universal Serial Bus (USB), or other type of serial interfaces. Additionally or alternatively, the interface 120 may be configured as a wireless interface such as via Wi-Fi, near field communication (NFC), 4G or 5G, BLUETOOTH®, or any other type of interfaces whether available now or invented hereafter as long as the features of the disclosed embodiments may be accomplished.

The processor(s) 106 may be any hardware device capable of executing instructions stored on memory 112 to accomplish the features of the disclosed embodiments. The processor 106 may include a microprocessor, a field programmable gate array (FPGA), an application-specific integrated circuit (ASIC), or other similar devices.

In some embodiments, such as those relying on one or more ASICs, the functionality described as being provided in part via software may instead be configured into the design of the ASICs and, as such, the associated software may be omitted. The processor 106 may be configured as part of the user device 104 (e.g., a laptop or desktop) or located at some remote location.

The memory 112 may be L1, L2, L3 cache, or RAM memory configurations. The memory 112 may include non-volatile memory such as flash memory, EPROM, EEPROM, ROM, and PROM, or volatile memory such as static or dynamic RAM, as discussed above. The exact configuration/type of memory 112 may vary as long as the processor(s) 106 can execute instructions for identifying cognitive decline.

The processor(s) 106 may be configured to execute one or more submodules that each present specific tests to the patient 108 and to analyze the provided drawing inputs. For example, the processor 106 may execute one or more drawing test submodules 122a, 122b, . . . 122n where n is the number of different tests that can be administered. Each of these drawing test submodules may be associated with an imagery analysis module 124a, 124b, . . . , 124m where m is the number of imagery analysis submodules. In some embodiments, m=n such as if there is an imagery analysis submodule associated with each drawing test submodule.

Each drawing test module 122a-n may be configured to administer a drawing test to the patient 108. The drawing test may promp the patient 108 to draw a certain type of object. The associated imagery analysis module 124a-m may apply one or more objective criterion in analyzing the received drawing input. Depending on whether, or to what extent the received drawing input satisfies the applied objective criterion, the scoring module 126 may calculate a score for the patient 108 indicating the likelihood of cognitive decline.

The database(s) 114, server(s) 116, or some combination thereof may store data associated with individual drawing tests, including the associated objective criterion. These components may also store data regarding tests completed by the patient 108 or other patients. These stored results may be used to track a patient's health over time.

The network(s) 118 may link the various components with various types of network connections. The network(s) 116 may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks 118 may also comprise, include, or interface to any one or more of a Wireless Application Protocol (WAP) link, a Wi-Fi link, a microwave link, a General Packet Radio Service (GPRS) link, a Global System for Mobile Communication (GSM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

As discussed above, the disclosed embodiments may identify cognitive decline of the patient 108 by administering one or more tests in which the patient 108 provides a drawing input. The patient 108 may be tasked with drawing a certain object for a certain test. Depending on errors in the drawing or otherwise how well the patient 108 performs on the test, the embodiments herein may identify one or more areas of cognitive decline of the patient 108.

The imagery analysis modules 124a-m may execute one or more imagery analysis techniques to analyze the received drawing input. These may include, for example, Speeded-Up Robust Features (SURF), Scale-Invariant Feature Transform (SIFT), Binary Robust Independent Elementary Features (BRIEF), Oriented FAST and Rotated BRIEF (ORB), or any other type of imagery analysis technique(s) whether available now or invented hereafter as long as the features of the described embodiments may be accomplished. These tools may analyze the drawing input as it compares to sample drawing inputs stored in one or more databases 114 or servers 116. These stored drawings may refer to drawings provided by other patients as well as model or “correct” examples of drawings.

In some embodiments, the received drawing input comprises raster image data, vector stroke data, and time-series input data captured while the patient 108 creates the drawing.. One or more of the imagery analysis modules 124a-m may preprocess the input by normalizing scale and orientation, segmenting the drawing from the background, and aligning the drawing to a reference template or model.. One or more of the imagery analysis modules 124-m may then extract geometric, spatial, and temporal features including stroke count, stroke order, drawing speed, pauses, pen pressure, line intersections, symmetry, closure, relative placement of components, occupied canvas area, and proximity of a copied figure to a presented model. These extracted features may be compared with reference drawings, normative datasets, and prior drawings from the same patient to generate conformance scores, anomaly scores, error categories, and longitudinal progression indicators.

In some embodiments, the drawing test module 122a may instruct the patient 108 to draw a clock. In some embodiments, the drawing test module 122a may issue a command to the patient device 110 to instruct the patient 108 to draw a clock showing a certain time. For example, the patient device 110 may instruct the patient 108 to “draw a clock, put in all the numbers, and draw the clock hands to show a time of 10 past 10.” These or similar types of instructions may be stored in and referenced from the database 114 or server 116.

These types of instructions assess multiple cognitive functions. To successfully complete this clock drawing task, the patient 108 must possess visuospatial ability to draw a round clock face with correctly spaced numbers, and must possess semantic memory to remember the layout of a clock and the meaning of “10 past 10.” The patient 108 must also possess executive function to plan and execute the placement of the two clock hands to show the requested time.

In this test, the patient 108 must understand and remember the instruction, and transform “10 past 10” into the visual layout of the clock hands. This engages the patient's frontal lobe networks for planning and working memory, as well as the parietal lobe networks for spatial processing. Specifically, the right parietal lobe is critical for visuospatial tasks. The frontal and dorsolateral prefrontal cortex are involved in the planning and sequencing to set the correct time. Exercises such as this clock-drawing exercise activate a broad cortical network and are therefore a sensitive indicator of cognitive integrity in those regions.

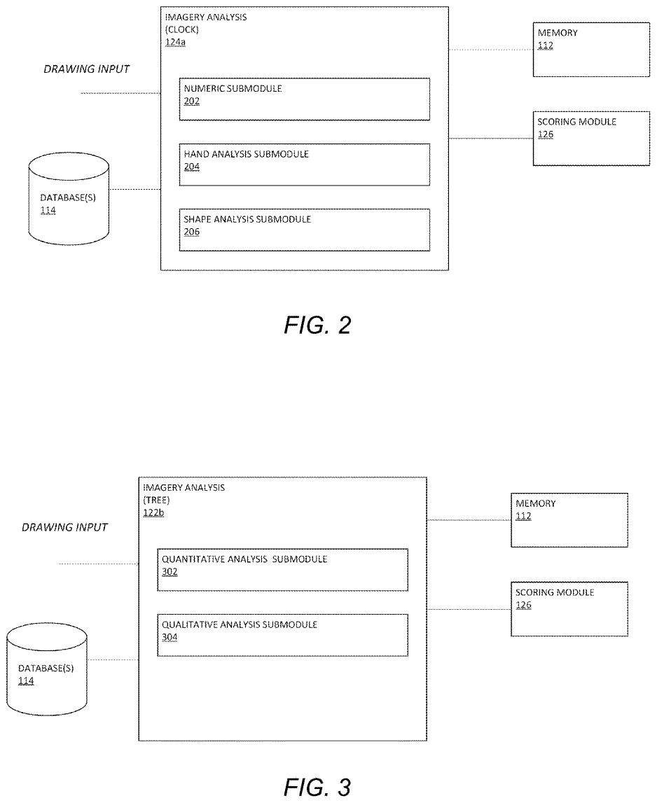

FIG. 2 illustrates the imagery analysis module 124a of FIG. 1 in accordance with one embodiment. As seen in FIG. 2, the imagery analysis module 124a may execute various submodules to analyze the received drawing input of the clock. Each submodule may analyze some different facet or feature of the clock. For example and without limitation, the imagery analysis module 124a may execute a numeric submodule 202, a hand analysis submodule 204, and a shape analysis submodule 206. Each submodule 202-06 may analyze some aspect of the drawing input from the patient 108 against one or more objective criterion to determine any errors.

The numeric submodule 202 may analyze the received drawing input to identify whether the patient has correctly drawn all numbers on the clock and in the correct place. The hand analysis submodule 204 may analyze the presence and placement of the clock hands. The shape analysis submodule 206 may analyze how well the patient 108 drew a circle to represent the clock face, for example.

The imagery analysis modules 124a-m may incorporate or otherwise rely on trained machine learning models to analyze the drawing inputs in real time. This yields several advantages over human or manual-based scoring. Specifically, the disclosed embodiments enable objectivity, granularity, and early detection of cognitive decline. This provides a consistent and objective analysis framework, and one that is not subject to human biases or tendency to overlook certain details (e.g., the placement of the clock hands in the clock drawing test).

In some embodiments, the system 100 employs one or more trained machine learning models, including convolutional neural networks, vision transformer models, object-detection models, segmentation models, Siamese similarity models, and ensemble classifiers, to identify drawing elements, classify error types, and estimate likelihood of cognitive decline. The models may be retrained or fine-tuned using newly collected de-identified drawings and clinician-validated labels so that performance improves over time across different devices, age groups, languages, and drawing styles. In some embodiments, lightweight models execute locally on the patient device 110 for near-real-time scoring while more computationally intensive models execute on a remote server such as server 116. The system 100 may use model compression, batching, caching, or hardware acceleration to reduce latency and increase processing speed.

A common error in the clock-drawing test is a stimulus-bound response, where the patient 108 literally places one hand at “10” and the other at “10” (or at 10” and “10+”). This is because the patient 108 focusses too heavily on the spoken numbers of the command and without proper interpretation. In some instances, a patient 108 may draw one hand at “10” and the other at “11,” thereby showing a time of 11:50 instead of 11:10. This error reveals the patient's inability to translate the instruction into a visual result. Patients with intact executive function, however, can inhibit the impulse to place a hand on the “10” for the minutes, and correctly position the hand at “2.”

Each submodule 202-06 may output a score, rank, vote, or otherwise some type of output indicating how well the patient 108 satisfied some objective criterion. For example, the numeric submodule 202 may output some score indicating how well the patient 108 was able to draw the numbers on the clock face. This may be based on how clearly the patient 108 wrote the numbers, how many numbers the patient drew, the placement of the numbers on the lock, etc. or some combination thereof. The hand analysis submodule 204 may output a score, rank, vote, or otherwise some output indicating how well the patient placed the clock hands to show a time of 10:10. This may be based on whether the patient 108 did in fact draw two hands, how well they placed the hour hand, how well they placed the minute hand, or some combination thereof. The shape analysis module 206 may output a score, rank, vote, or otherwise some output indicating whether the patient 108 correctly drew a clock shape, such as a circle represent the face of the clock.

Referring back to FIG. 1, as another test example, the drawing test module 122b may execute instructions to instruct the patient 108 to draw a tree. In some embodiments, the drawing test module 122b may cause the patient device 110 to instruct the patient 108 to “draw a tree, any kind of tree you like.” Unlike the clock-drawing test, this type of prompt is an open-ended task with no further guidance or even time limit. This type of prompt yields a rich set of data, primarily due to the quality and quantity of detail the patient 108 provides.

To perform this task, the patient 108 must retrieve the generic concept of a tree from their semantic memory, which is associated with their temporal lobes. If their memory or knowledge of what elements constitute a tree is poor, they will have difficulty in drawing a tree or feature thereof.

This task also requires visuospatial skills and constructional abilities like those required to draw a complex figure. The parietal lobes are responsible for handling the spatial layout of this drawing, such as by ensuring that the tree trunk and the crown are connected, placing roots at the bottom of the tree, and drawing the tree with proper proportions. The frontal lobe and, in particular, the patient's prefrontal cortex, contributes to the planning and organization needed to make the drawing such as where to start, how to size the tree, in what order to add features, etc.

FIG. 3 illustrates the imagery analysis module 124b in accordance with one embodiment. The imagery analysis module 124b may execute one or more submodules to analyze the received tree drawing input such as, but not limited to, a quantitative analysis submodule 302 and a qualitative analysis submodule 304.

The quantitative analysis submodule 302 may execute one or more image analysis procedures to identify quantitative aspects of the received drawing input. The quantitative analysis submodule 302 may measure dimensions of the tree drawing input such as the height and width of the trunk, the height and width of the crown, the tilt angle of the trunk, the sizes of branches, the sizes of leaves, etc. The quantitative analysis submodule 302 may also compute ratios of components of the tree, such as the trunk-to-crown ratio (i.e., trunk height divided by the crown height), as well as the proportion of the drawing canvas occupied by the drawing. These calculated features provide objective data points regarding the patient's drawing input.

The qualitative analysis submodule 304 may identify elements of the tree such as the crown, trunk, roots at the base or below ground, leaves, branches, or the like. A healthy or otherwise cognitively intact patient will typically draw a tree with a distinct crown and a fluffy outline to represent foliage or individual branches. They may also illustrate roots or add some branches or a ground line.

Non-healthy patients or otherwise patients suffering from some level of cognitive decline may instead produce a rudimentary tree. For example, patients with Alzheimer's disease may draw just a simple tree trunk with no branches or roots. The qualitative analysis module 304 may identify not only whether elements are present, but also how well-formed they are.

The imagery analysis module 124b may reference memory 112 or databases 114 for data regarding how to interpret the patient's drawing. Patients with Alzheimer's disease tend to draw smaller trees overall (i.e., their drawings take up much less of the page compared to drawings made by healthy patients). An Alzheimer's patient may draw a short trunk, small branches, and a small group of leaves. Elements of this size may reflect both spatial scaling issues and apathy or even hesitance in making the drawing. Patients with Alzheimer's disease also tend to make drawings with a higher trunk-to-crown ratio in which the tree canopy or crown is small compared to the trunk. This is because patients with Alzheimer's draw fewer branches and shrunken or less detailed leafy area.

Patients with frontotemporal dementia (for simplicity, “FTD”) may produce trees that are more expansive. They may draw a tree using a wider space on their drawing canvas as compared with Alzheimer's patients. A drawing by an FTD patient may have a broad crown or extensive branching, which may still appear peculiar if they also drew a large trunk or exaggerated ground line. This may be because FTD patients may approach the task impulsively, such as by first sketching a big outline of the tree, but not filling in fine details.

Patients with vascular dementia (for simplicity, “VaD”), especially those who have suffered from strokes that affected their right parietal or frontal regions, may draw small or simple trees similar to those made by Alzheimer's patients. Patients with a lesser impairment of VaD may draw nearly normal-sized trees with just slight deficits such as a missing root or an asymmetrical crown.

Patients with a mild cognitive impairment, such as an early stage of cognitive decline, tend to produce tree drawings that are intermediate in size and provide an amount of detail between that provided by healthy patients and those with dementia. The disclosed embodiments may leverage these differences by evaluating a drawing input against known patterns. For example, a drawing with a high trunk-to-crown ratio and a small overall size could suggest the patient 108 has Alzheimer's disease, whereas a large drawing with odd proportions could suggest the patient 108 has FTD.

As another drawing test example, a drawing test module may execute instructions to instruct the patient 108 to draw a cube. In some embodiments, the patient device 110 may present an image of a cube, and instruct the patient 108 to copy the cube in an area next to the presented cube image. The cube test presents a challenge to the patient 108 as it requires their brain to interpret a three-dimensional “3D” object on a two-dimensional “2D” plane, and then reproduce that interpretation.

The cube test is essentially a test of the dorsal visual stream, which is the part of the brain that processes spatial relationships. By observing whether, and to what extent, a provided cube drawing is distorted, the present embodiments can determine which cognitive processes are failing. For example, a systematically rotated or tilted cube may indicate a visuospatial bias or neglect. As another example, a cube drawn with hesitancy or with erasures may indicate executive self-monitoring issues.

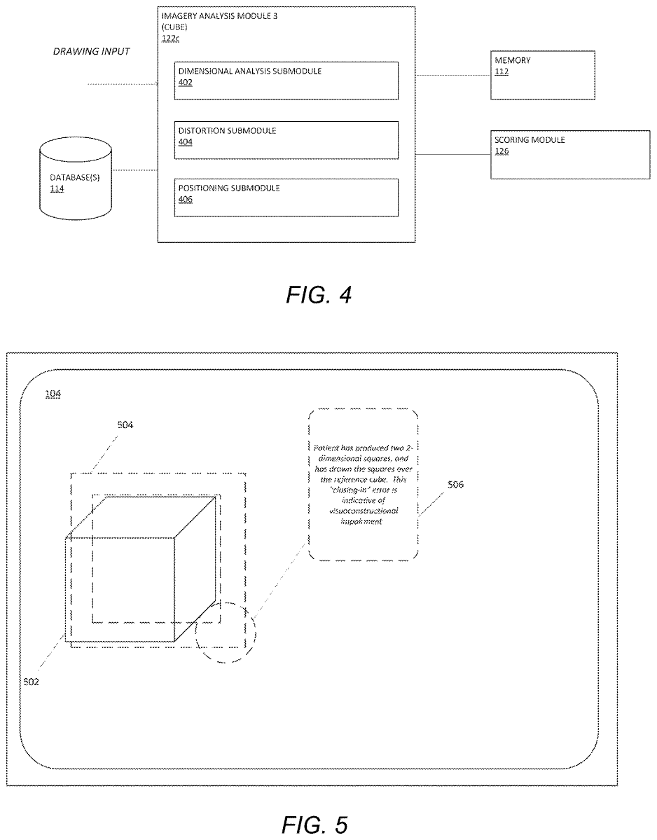

FIG. 4 illustrates an imagery analysis module 124c (not shown in FIG. 1) in accordance with one embodiment. The imagery analysis module 124c may include or otherwise execute one or more submodules such as a dimensional analysis submodule 402, a distortion submodule 404 and a positioning submodule 406.

The dimensional analysis submodule 402 may execute one or more imagery analysis procedures to detect if the patient 108 provided a simplified version of a cube, such as by drawing just a square or two unconnected squares. In other words, the dimensional analysis submodule 402 detects if the patient 108 was unable to create a 3D representation, and instead provided one or more 2D shapes.

The distortion submodule 404 may identify the existence and degree of any distortion(s) in the patient's provided cube. For example, the patient 108 may have drawn a cube with angled sides, misplaced lines, extra lines, extra faces, depth lines that are not parallel, lines that don't close properly, or the like.

The distortion submodule 404 may also detect whether a patient 108 has drawn the cube extremely close to the presented cube or even overlapping the presented cube. This “closing in” error is common with patients with Alzheimer's disease. The closing-in error is not caused by eyesight, but instead a cognitive occurrence believed to relate to visuospatial disorientation or a compensatory strategy. The patient's brain relies on the presented cube for guidance because they have reduced spatial working memory.

The positioning submodule 406 may analyze the placement of the patient's drawing relative to the presented cube on the screen of the patient device 110. In some embodiments, the test may instruct the patient 108 to draw their cube adjacent to a reference point. The positioning submodule 406 may identify the position of the patient's cube with respect to the reference point.

The three tests discussed above, whether considered alone or in some combination, provide a wealth of data regarding a patient's cognitive decline. The tree drawing test determines whether the patient 108 can spontaneously create a drawing, the clock drawing test measures how well the patient 108 interprets an instruction, and the cube drawing test measures how well the patient 108 can copy a complex visual stimulus.

However, the features of the claimed embodiments are not limited to any particular or specific drawing test. Although the present application discusses three specific drawing-related tasks, more than or less than three drawing tests may be administered to a patient. These may include tests in addition to or in lieu of those described above.

As seen in FIGS. 1-4, a scoring module 126 may receive output from one or more of the image analysis modules 124a-m. The scoring module 126 may assign score(s) representing the likelihood of cognitive decline in a variety of ways.

For example, the scoring module 126 may assign point(s) to each error detected by the respective imagery analysis modules 124a-m. Similarly, the scoring module 126 may assign a greater number of points the greater the extent of a detected error. For example, a cube that is distorted by some measurable aspect(s) may receive a higher score than a cube that is less distorted by the same measurable aspect(s).

Alternatively, the scoring module 126 may assign points based on each detected correct aspect of a drawing. In other words, healthy individuals would likely receive high scores. Patients experiencing cognitive decline would receive lower scores.

The scoring module 126 may also assign weights to scores associated individual attributes of a drawing, such as if a certain type of error is strongly related with cognitive decline. For example, the “closing in” error in the cube drawing test is strongly associated with visuoconstructional impairment. Accordingly, the scoring module 126 may assign detection of the “closing in” feature a high weight such that it produces a score indicating cognitive decline and, in particular, visuoconstructional impairment.

The user device 104 may be configured to output a summary of the patient's score. The score may be presented as a single numerical value, multiple numeric values, text, graph, or any other type of data structure as long as it can convey to the user the results and meaning of the patient's test results. In some embodiments, the user device 104 may present the drawings provided by the patient 108, as well as highlights or annotations associated with the drawing(s) to indicate errors that contributed to the patient's score(s).

FIG. 5 presents the user device 104 of FIG. 1 showing a results pane in accordance with one embodiment. The user device 104 may present data to the user 102 regarding how well a patient performed on the cube drawing test. The user device 104 may present the model cube 502, as well as the patient's cube 504. As seen in FIG. 5, the patient 108 in this case has drawn their cube substantially “over” the model cube 502. The user device 104 also presents an annotation window 506, which provides the user with an explanation of what the drawing shows and what it suggests. In this case, the patient has made the “closing in” error, and the annotation 506 indicates this is indicative of visuoconstructional impairment. The user device 104 may also present a score indicating the likelihood of cognitive decline of the patient. The user device 104 may similarly present other types of results or summary pages for other drawings.

Referring back to FIG. 1, the treatment plan generation module 128 may consider the score(s) outputted by the scoring module 126 to generate one or more treatment plans. The treatment plan may be based on, for example, the type(s) of errors detected in the drawing input(s), as well as the severity of those errors.

In some embodiments, the treatment plan generation module 128 generates a personalized follow-up plan based on one or more of the composite score, the detected error category, severity of impairment, and longitudinal changes across repeated assessments. The generated plan may include one or more recommendations for repeat screening at a defined interval, referral to a primary care physician, neurologist, or neuropsychologist, caregiver education, medication review, safety counseling, lifestyle interventions, cognitive training exercises, or additional diagnostic testing. In some embodiments, the treatment plan is presented as clinical decision support for review, modification, and approval by a healthcare provider before implementation.

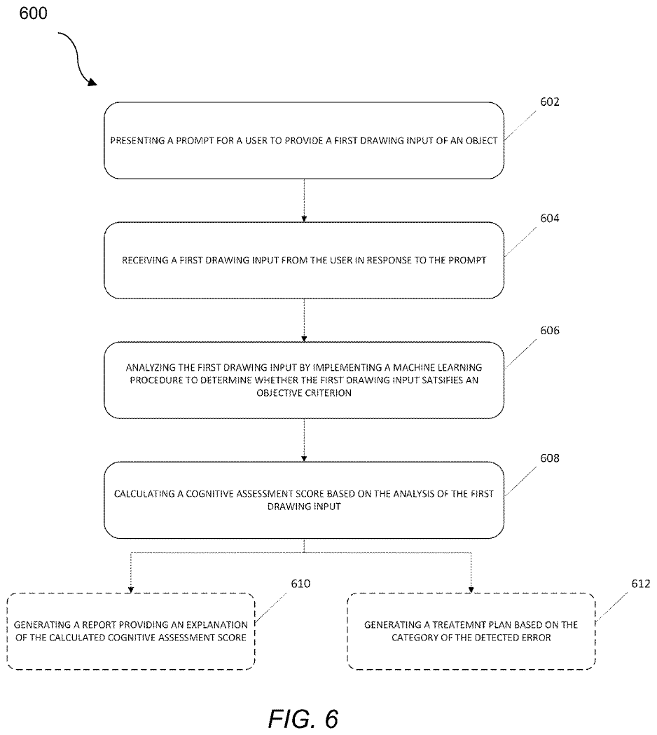

FIG. 6 depicts a flowchart of a method 600 for identifying cognitive decline in accordance with one embodiment. The system 100 of FIG. 1 may perform one or more of the steps of method 600.

Step 602 involves presenting, via a user interface executing on a digital device, a prompt for a user to provide a first drawing input of an object. The digital device may be similar to the patient device 110 of FIG. 1. The digital device may refer to, for example, and without limitation, a smart phone, computer screen, tablet, web interface, or any other type of device that allows the patient to receive and instruction and provide the requested drawing input. The first drawing input may refer to any of the aforementioned drawing tests, such as the cube drawing test, the clock drawing test, or the tree drawing test.

Step 604 involves receiving, via the user interface, a first drawing input from the user in response to the prompt. Step 606 involves analyzing the first drawing input by implementing a machine learning procedure to determine whether the first drawing input satisfies an objective criterion. The objective criterion may depend on the type of drawing test administered. For example, for the clock drawing test, the objective criterion may refer to whether the patient drew all numbers of the lock, drew the clock hands, placed the clock hands in the correct position, etc. For the cube drawing test, the objective criterion may refer to whether the patient drew a 3D shape, whether the patient positioned their cube in the correct spot, etc. For the tree drawing test, the objective criterion may refer to the size of the tree or components thereof, ratios of components, the number of branches they patient drew, or the like.

Step 608 involves calculating a cognitive assessment score based on the analysis of the first drawing input, wherein the cognitive assessment score indicates a likelihood of cognitive decline of the user. The scoring module 126 of FIG. 1 may consider outputs from one or more imagery analysis modules in determining whether the patient is at least likely experimenting cognitive decline.

Step 610 involves generating a report providing an explanation of the calculated cognitive assessment score. For example, the user interface may present to the user a summary of the patient's test results, including an explanation of the error and what it suggests. Step 612 involves generating a treatment plan based on the category of the detected error.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for identifying cognitive decline, the method comprising:
presenting, via a user interface executing on a digital device, a prompt for a user to provide a first drawing input of an object;
receiving, via the user interface, a first drawing input from the user in response to the prompt;
analyzing the first drawing input by implementing a machine learning procedure to determine whether the first drawing input satisfies an objective criterion; and
calculating a cognitive assessment score based on the analysis of the first drawing input, wherein the cognitive assessment score indicates a likelihood of cognitive decline of the user.

2. The method of claim 1 wherein analyzing the first drawing input comprises evaluating at least one of visuospatial ability, semantic memory, or executive function based on characteristics of the first drawing input.

3. The method of claim 1 further comprising generating a report providing an explanation of the calculated cognitive assessment score.

4. The method of claim 3, wherein the generated report includes a highlighted portion of the first drawing input and an annotation accompanying the highlighted portion.

5. The method of claim 1 further comprising generating a treatment plan for the user based on the calculated cognitive assessment score.

6. The method of claim 1 wherein analyzing the first drawing input includes:
detecting an error in the first drawing input, and
categorizing the detected error, wherein the calculated cognitive assessment score is based on the category of the detected error.

7. The method of claim 6, further comprising generating a treatment plan based on the category of the detected error.

8. The method of claim 1 wherein analyzing the first drawing input includes:
presenting a model of the object to the user via the user interface, and
detecting conformance of the first drawing input to the model, wherein the generated cognitive assessment score is based at least in part on the conformance of the first drawing input to the model.

9. The method of claim 1 further comprising:
presenting at least a second prompt for the user to provide a second drawing input of a second object,
receiving a second drawing input from the user,
analyzing the second drawing input, wherein each of the first drawing input and the second drawing input are designed to test one or more of visuospatial ability, semantic memory, or executive function based on characteristics of each drawing input.

10. A system for identifying cognitive decline, the system comprising:
a user interface executing on a digital device and configured to:
present a prompt for a user to provide a first drawing input of an object, and
receive a first drawing input from the user in response to the prompt; and
one or more processors executing instructions stored on memory to:
analyze the first drawing input by implementing a machine learning procedure to determine whether the first drawing input satisfies an objective criterion, and
calculate a cognitive assessment score based on the analysis of the first drawing input, wherein the cognitive assessment score indicates a likelihood of cognitive decline of the user.

11. The system of claim 10 wherein the one or more processors analyze the first drawing input by evaluating at least one of visuospatial ability, semantic memory, or executive function based on characteristics of the first drawing input.

12. The system of claim 10 wherein the one or more processors are further configured to generate a report providing an explanation of the calculated cognitive assessment score.

13. The system of claim 12 wherein the generated report includes a highlighted portion of the first drawing input and an annotation accompanying the highlighted portion.

14. The system of claim 10 wherein the one or more processors are further configured to generate a treatment plan for the user based on the calculated cognitive assessment score.

15. The system of claim 10 wherein the one or more processors analyze the drawing input by:
detecting an error in the drawing input, and
categorizing the detected error, wherein the calculated cognitive assessment score is based on the category of the detected error.

16. The system of claim 15 wherein the one or more processors are further configured to generate a treatment plan for the user based on the category of the detected error.

17. The system of claim 10 wherein the one or more processors analyze the drawing input by:
presenting a model of the object to the user via the user interface, and
detecting conformance of the first drawing input to the model, wherein the generated cognitive assessment score is based at least in part on the conformance of the first drawing input to the model.

18. The system of claim 10 wherein the user interface is further configured to:
present at least a second prompt for the user to provide a second drawing input of a second object, and
receive a second drawing input from the user, and the one or more processors are further configured to analyze the second drawing input, wherein each of the first drawing input and the second drawing input are designed to test one or more of visuospatial ability, semantic memory, or executive function based on characteristics of each drawing input.

19. A computer program product for identifying cognitive decline, the computer program product comprising computer executable code embodied in one or more non-transitory computer readable media that, when executing on one or more processors, performs the steps of:
presenting, via a user interface executing on a digital device, a prompt for a user to provide a first drawing input of an object;
receiving, via the user interface, a first drawing input from the user in response to the prompt;
analyzing the first drawing input by implementing a machine learning procedure to determine whether the first drawing input satisfies an objective criterion; and
calculating a cognitive assessment score based on the analysis of the first drawing input, wherein the cognitive assessment score indicates a likelihood of cognitive decline of the user.

20. The computer program product of claim 19 wherein analyzing the first drawing input comprises evaluating at least one of visuospatial ability, semantic memory, or executive function based on characteristics of the drawing input.
